(19) United States
(12) Patent Application Publication
(10) Pub. No.: US 2026/0242741 A1
(43) Pub. Date: Aug. 20, 2026

(54) METHOD FOR MAKING VESICLES

(71) Applicant: CAPRICOR, INC., San Diego, CA (US)

(72) Inventors: Kristi ELLIOTT, Encinitas, CA (US); Minghao SUN, San Diego, CA (US); Michael LECLAIRE, San Diego, CA (US); Justin NICE, San Diego, CA (US)

(21) Appl. No.: 19/118,767

(22) PCT Filed: Oct. 5, 2023
(86) PCT No.: PCT/US2023/076041

§ 371 Date: Apr. 5, 2025

Related U.S. Application Data
(60) Provisional application No. 63/413,806, filed on Oct. 6, 2022.
(60) Provisional application No. 63/536,639, filed on Sep. 5, 2023.

(51) Int. Cl. C12N 5/071; B01D 21/26; B01D 61/14; C12N 5/077

(52) U.S. Cl. CPC C12N 5/0686; B01D 21/262; B01D 61/145; C12N 5/0657

(57) ABSTRACT

Disclosed generally are methods of purifying and concentrating exosomes produced in cell culture. Disclosed specifically are methods of purifying and concentrating native or engineered exosomes produced in cell culture by cell culture clarification, filtration, concentration by filtration, removal from contaminants by molecular sieve, and additional concentration by filtration to a concentration on the order of trillions (E12) of exosomes per milliliter.

### REFERENCE TO RELATED APPLICATIONS

This application claims the benefit of priority dates of U.S. Provisional Application No. 63/413,806, filed Oct. 6, 2022, and U.S. Provisional Application No. 63/536,639, filed Sep. 5, 2023, the contents of which are incorporated herein by reference in their entireties.

### BACKGROUND

Exosomes are small (~30-200 nm) membrane-bound vesicles produced in the endosome compartment and secreted by cells and take up by recipient cells. Exosomes contain and transport natural cargos such as nucleic acids, proteins, lipids, amino acids, and metabolites. Exosomes can be engineered to deliver payloads to target cells, and are therefore an important new pharmacological modality capable of delivering therapeutic effects, medicaments, vaccines, and other therapeutic or prophylactic cargos. For a review of exosomes and their therapeutic potential, see e.g., Liang et al., Theranostics, 2021 Jan. 1;11 (7): 3183-319; Pegtel and Gould, Annu Rev Biochem, 2019 Jun. 20; 88:487-514; Kalluri and LeBleu, Science, 2020 Feb. 7;367 (6478): eaau6977.

The manufacture, purification, isolation, and formulation of exosomes for commercial pharmaceutical use is complicated and not well worked out. Currently, several methods for producing exosomes at lab-scale are available. Those methods include, e.g., (i) ultracentrifugation, in which exosomes are pelleted at ~100,000×g for ~2 hour, (ii) immunoaffinity purification, in which exosomes are captured on magnetic beads or other matrix material decorated with cognate binding elements, and (iii) polymer precipitation. Those methods are incapable of generating sufficient quantities or pure exosomes and are fraught with other drawbacks such as protein contamination, undesirable buffers, and clinically problematic polymer contamination.

While bench or lab-scale methods production of exosomes are currently known in the art, cost effective and scalable commercial processes that enable the large-scale production of exosomes having clinically relevant purity, concentration, and volume (i.e., >1E12 exo/mL and ≥40 ml) are still sorely needed.

### SUMMARY

Disclosed is a scalable process for making, concentrating, isolating, and/or purifying large quantities of vesicles at high concentration. Vesicles include extracellular vesicles (EVs) including microvesicles (MVs) and exosomes (XOs).

Here, the process includes (i) obtaining a clarified cell culture supernatant containing vesicles and having a first volume, (ii) cross filtering the clarified cell culture supernatant to produce a first retentate containing the vesicles, (iii) sieving the first retentate and collecting the fraction that contains the vesicles, and (iv) cross filtering the fraction collected from the sieve and collecting a second retentate containing the vesicles. Optionally, the second retentate is frozen and then thawed to cause certain impurities to precipitate. The precipitate is removed from the vesicle suspension by centrifugation, and the clarified supernatant is filter sterilized, such as, e.g., through a 0.2-micron filter.

The clarified cell culture supernatant can be obtained by centrifuging a suspension cell culture at a relatively low force to remove cells, removing the cells, and then centrifuging the remaining supernatant at a higher force to produce a clarified cell culture supernatant. Here, the centrifugal force used to remove the cells can be about 200-400×g and the subsequent force used to clarify the supernatant can be about 2,000-4,000×g.

The vesicle purification steps are designed to minimize vesicle loss while significantly increasing vesicle concentration and purity. Here, the vesicles are enriched about 100-fold to 400-fold from the starting clarified cell culture supernatant to the second retentate. Here, the concentration of vesicles in the second retentate or filter sterilized supernatant is about 1E11-1E13 vesicles/mL or greater, and in volumes on the order of tens of milliliters to multiples of liters, for example 40 mL to 160 mL or greater. Useful vesicles may have a diameter of about 3-200 nm, such as in the case for exosomes.

The first and/or second cross-filtering(s) can be performed across a 750 kDa filter having, e.g., an approximate pore size of about 13 nm. The 750 kDa filter can be a hollow fiber filter and the cross-filtering can be performed via tangential flow filtration (TFF) using a system, such as e.g., ÄKTA flux tangential flow filtration system (Cytiva, Marlborough, MA). Here, vesicles (including exosomes) remain in the retentate fraction given their larger-than-13 nm size, while small proteins and other contaminants pass through into the permeate.

After the first cross-filtering, the first retentate can be subjected to molecular sieve (e.g., size exclusion) chromatography and the excluded fraction, which elutes first and contains the vesicles, is collected. Here, bind-elute or simple molecular sieve matrix may be used, such as, e.g., Sephacryl™ S HR chromatography media (Cytiva, Marlborough, MA).

The vesicles can be further concentrated by subjecting the fraction collected from the molecular sieve to cross-filtering, such as, e.g., by TFF across a 750 kDa (~13 nm) cut-off filter. Here, the vesicles can be concentrated an additional 4-fold to 10-fold or greater than the vesicle concentration in the collected fraction.

The post molecular sieve cross-filtered vesicle suspension can be clarified or further purified by removing contaminants by freezing and thawing the suspension, centrifuging the suspension, and removing and retaining the supernatant while avoiding the pellet that contains the contaminants. The contaminate-reduced vesicle suspension may by sterilized by 0.2-micron filtration.

The cells from which the vesicle-containing clarified cell culture supernatant is obtained can be any cells, including more generally mammalian cells or more specifically human cells, such as, e.g., cardiosphere-derived cells (CDCs) or HEK293 or their derivatives. Here, the cells secrete vesicles, such as, e.g., exosomes, into the culture supernatant.

The cells may be engineered to load cargo (e.g., nucleic acids, modified nucleic acids, small molecules drugs, proteins, large molecule biologics such as antibodies and antibody fragments, etc.) into the vesicles. Also, the purified vesicles may be loaded exogenously.

The cells may be engineered to express a fusion protein that is sorted or otherwise loaded into the vesicles and functions as a targeting moiety to enable the preferential binding or targeting of the vesicles to a specified target molecule, cell, or tissue. Here as well, the vesicles may also be loaded (endogenously or exogenously) with cargo.

### DRAWINGS

FIG. 1 is a flow diagram depicting process steps and products for producing purified vesicles from a cell suspension.

FIG. 2 is a flow diagram depicting process steps and products for producing purified vesicles from a cell suspension.

FIG. 3 is a flow diagram depicting lab scale [300], scalable scale [310], and large scale GMP exosomes isolation processes.

FIG. 4 is a graphic depicting the principle of tangential flow filtration (TFF) as applied to concentrating exosomes that are maintained in the retentate (larger circles). Smaller circles represent permeate particles/solutes.

FIG. 5 is a line graph depicting protein absorbance at 214 nm as a function of eluted volume in milliliters for a size exclusion column loaded with exosomes and undesirable contaminants. The first peak demarked by dotted lines represents the eluted volume containing exosomes as determined by subsequent nanoparticle tracking analysis (NTA).

FIG. 6 are transmission electron micrographs at the nanometer scale. Panel A depicts exosomes in pooled peak #1 from exosome-loaded SEC column as represented in FIG. 5. Panels B and C depict contents of pooled peaks 2 and 3 subsequent to the pooled peak #1. Panel B (first pooled peak after the initial exosome-containing peak) shows the presence of some 40 nm undefined material and 50-60 nm circular particles, but nothing the size of exosomes. Panel C shows the contents of pooled peaks 3, 4, and 5 subsequent to polled peaks 1 and 2. The scale bars in panels A and B are 200 nm. The scale bar of panel C is 500 nm.

FIG. 7A is a TEM micrograph showing purified and concentrated exosome (bar=~100 nm). Inset is a blow-up of one exosome.

FIG. 7B is a line graph depicting normalized amount of exosomes as a function of size in nanometers.

FIG. 7C is a histogram depicting the percent expression of CD81 as a function of three different lots of exosomes.

FIG. 8 is a Western blot depicting specific protein expression as a function of cells or cell factions. Column one is size markers. Column 2 is protein extracted from engineered cells from which exosomes are produced. Columns 3 and 4 are protein extracted from two different lots of exosomes produced from the engineered cells represented in column 2.

### DETAILED DESCRIPTION

Disclosed are embodiments of a method for making, isolating, and/or purifying vesicles, including exosomes, from cells. The method is applicable to purifying any vesicle from any cell type, including but not limited to exosomes secreted by cardiosphere-derived cells (CDCs) (see, e.g., Marbán et al., U.S. Patent Application Publication US 2022 0119813 A1), engineered exosomes, engineered 293F exosomes, exosomes expressing antigens, such as 293F engineered exosomes displaying SARS-COV2 spike or nucleocapsid, influenza hemagglutinin, or respiratory syncytial virus fusion protein, and the like (see, e.g., co-pending applications Sun et al., “SARS-COV-2 IMMUNOGENIC COMPOSITIONS AND METHODS,” PCT/US23/72876; Sun et al., “COMBINATION EXOSOMAL IMMUNOGENIC COMPOSITIONS AND METHODS,” PCT/US23/75343. The disclosed embodiments and examples are illustrative of the invention and do not limit the scope of the invention.

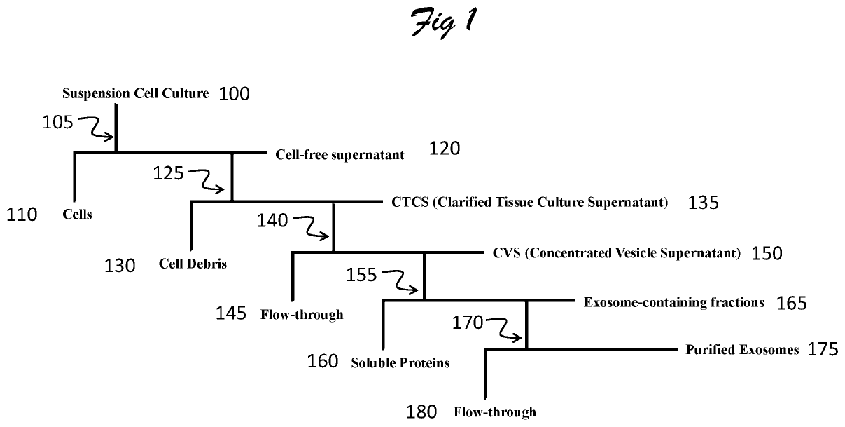

Turning to FIG. 1, the method includes centrifuging at a relatively low force 105 a suspension cell culture 100, which contains cell-secreted vesicles, including exosomes. The centrifuging 105 may be performed at a force of 300×g. After centrifuging 105, the formed cell pellet 110 may be discarded, and the cell-free supernatant 120, which contains the secreted vesicles, is subjected to further processing.

The cell-free supernatant 120 is centrifuged at relatively higher force 125 to remove cell debris 130 to produce a clarified supernatant 135 that contains the secreted vesicles. The centrifuging 125 may be performed at a force of 3,000×g. After centrifuging 125, the formed cell debris pellet 130 may be discarded, and the clarified supernatant 135, which contains the secreted vesicles, is subjected to further processing.

The clarified supernatant 135 is subjected to tangential flow filtration (TFF) 140 across a filter with a pore size that retains exosomes in the concentrated vesicle supernatant (CVS) retentate 150 while allowing small proteins and other contaminants to be removed with the flow through permeate 145. The TFF filter can be a hollow fiber filter with a cut-off size of about 13 nm diameter or about 750 kDa. Here, up to 99.98% of protein content can be removed from the clarified supernatant 135 into the flow-through permeate 145 to produce a purer and more concentrated exosome/vesicle CVS retentate 150. Here, at least 66% or more of the vesicles are retained in the CVS retentate 150, which is further processed 155 to remove soluble proteins and other contaminants 160.

The CVS retentate 150 is applied to a molecular sieve (e.g., size exclusion chromatography) 155. The excluded volume (void), which may correspond to the first protein absorbance peak that comes off the column, contains the vesicles (the exosome-containing fractions 165). Soluble proteins and other smaller molecular entities (collectively 160) remain on the column or within the sieve matrix and can optionally be eluted prior to any step of matrix regeneration.

The exosome-containing fractions 165 is subjected to tangential flow filtration (TFF) 170 across a filter with a pore size that retains exosomes in a second concentrated vesicle supernatant (sCVS) retentate containing purified exosomes 175 while allowing small proteins and other contaminants to be removed with the flow through permeate 180. The TFF filter can be a hollow fiber filter with a cut-off size of about 13 nm diameter or about 750 kDa. Here, the exosomes are concentrated into the retentate containing purified exosomes 175 while excess buffer and smaller residual fragments and contaminants go into the second flow-through permeate 180 to produce a purer and more concentrated exosome/vesicle second CVS retentate 175. Here, at least 66% or more of the vesicles are retained in the second CVS retentate 175.

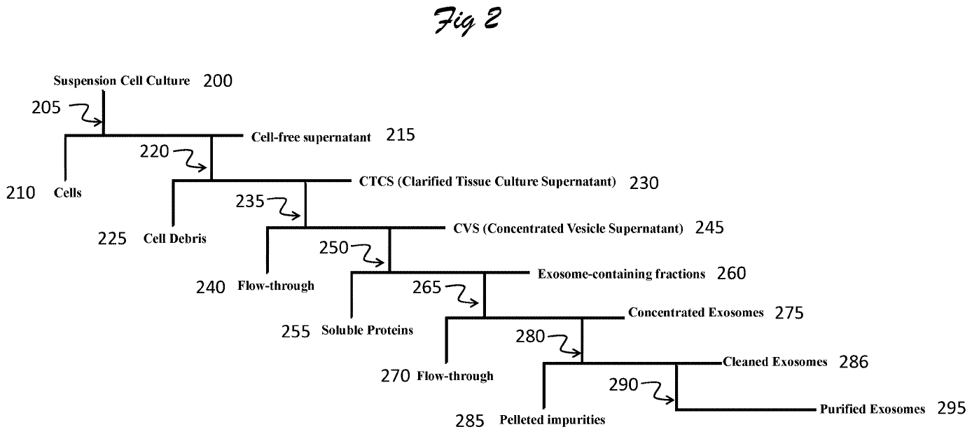

Turning to FIG. 2, the method includes centrifuging at a relatively low force 205 a suspension cell culture 200, which contains cell-secreted vesicles, including exosomes. The centrifuging 205 may be performed at a force of 300×g. After centrifuging 205, the formed cell pellet 210 may be discarded, and the cell-free supernatant 215, which contains the secreted vesicles, is subjected to further processing.

The cell-free supernatant 215 is centrifuged at relatively higher force 220 to remove cell debris 225 to produce a clarified supernatant 230 that contains the secreted vesicles. The centrifuging 220 may be performed at a force of 3,000×g. After centrifuging 220, the formed cell debris pellet 225 may be discarded, and the clarified supernatant 230, which contains the secreted vesicles, is subjected to further processing.

The clarified supernatant 230 is subjected to tangential flow filtration (TFF) 235 across a filter with a pore size that retains exosomes in the concentrated vesicle supernatant (CVS) retentate 245 while allowing small proteins and other contaminants to be removed with the flow through permeate 240. The TFF filter can be a hollow fiber filter with a cut-off size of about 13 nm diameter or about 750 kDa. Here, up to 99.98% of protein content can be removed from the clarified supernatant 230 into the flow-through permeate 240 to produce a purer and more concentrated exosome/vesicle CVS retentate 245. Here, at least 66% or more of the vesicles are retained in the CVS retentate 245, which is further processed 250 to remove soluble proteins and other contaminants 255.

The CVS retentate 245 is applied to a molecular sieve (e.g., size exclusion chromatography) 250. The excluded volume (void), which may correspond to the first protein absorbance peak that comes off the column, contains the vesicles (the exosome-containing fractions 260). Soluble proteins and other smaller molecular entities (collectively 255) remain on the column or within the sieve matrix and can optionally be eluted prior to any step of matrix regeneration.

The exosome-containing fractions 260 is/are subjected to tangential flow filtration (TFF) 265 across a filter with a pore size that retains exosomes in a second concentrated vesicle supernatant (sCVS) retentate containing concentrated exosomes 275 while allowing small proteins and other contaminants to be removed with the flow through permeate 270. The TFF filter can be a hollow fiber filter with a cut-off size of about 13 nm diameter or about 750 kDa. Here, the exosomes are concentrated into the retentate containing concentrated exosomes 275 while excess buffer and smaller residual fragments and contaminants go into the second flow-through permeate 270 to produce a purer and more concentrated exosome/vesicle second CVS retentate 275. Here, at least 66% or more of the vesicles are retained in the second CVS retentate 275.

The second CVS retentate containing the concentrated exosomes 275 is frozen and thawed and centrifuged 280 to pellet precipitated impurities 285 and the supernatant containing cleaned exosomes 286 is retained.

The cleaned exosomes 286 are then sterile filtered 290, such as, e.g., through a 0.2 μm filter, to produce the purified exosomes 295.

In some embodiments, a method for producing concentrated and pure exosomes includes a first step of producing exosomes from cells, which are secreted into the cell culture supernatant, a next step of removing cells and debris from the cell culture, a next step of concentrating the exosomes, a next step of removing protein from the concentrated exosomes, and next step of concentrating the protein-reduced exosomes suspension, and then a step of cleaning the exosome-containing product. The cleaned product contains exosomes at a concentration of 1E10-1E14 particles per mL, 1E11-1E13 particles per mL, about 1E12 particles per mL, or about 1-4E12 particles/mL.

In some embodiments, the method for producing exosomes produces a batch of about 4E12 purified exosomes to about 140E12 purified exosomes from about 1 liter to about 30 liters of cell culture. In some embodiments, the method for producing exosomes produces a batch of about 140E12 purified exosomes from a 30-liter cell culture. In some embodiments, the method for producing exosomes produces a batch of about 4E12 purified exosomes from a 1-liter cell culture. In some embodiments, the method for producing exosomes produces a batch of about 40E12 purified exosomes from a 4-liter cell culture.

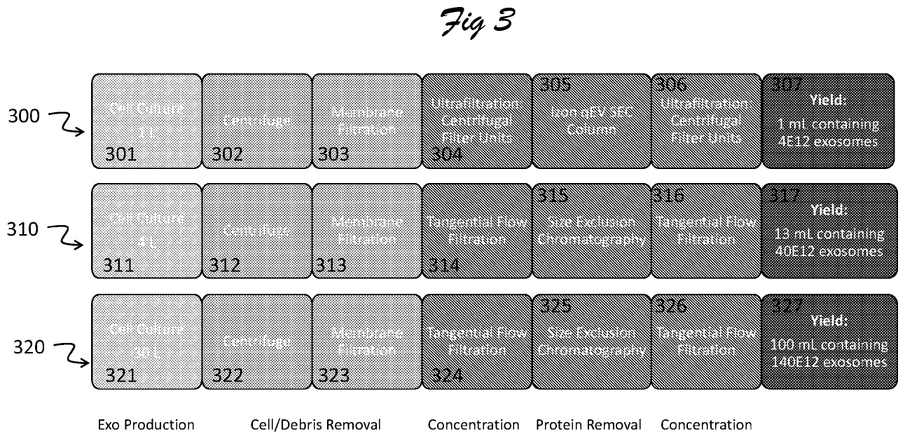

In one embodiment, the method is lab scale. FIG. 3 summarizes the lab scale process [300]. Here, a 1-liter cell culture [301] is cultured and exosomes are produced and secreted into the culture medium (exosome production). Cells and cell debris are then removed from the culture medium by centrifugation [302] followed by membrane filtration [303]. The exosomes are concentrated from the culture media sans cells and cell debris by ultrafiltration [304] such as by e.g., centrifugal filter units such as e.g., Centricon Plus-70 with a 100 kDa cut-off. Protein is

then removed from the exosome concentrate by size exclusion [305] such as by e.g., an Izon qEV SEC column. After protein removal, the exosomes are concentrated by ultrafiltration such as e.g., by using centrifugal centrifugation units. Here, the yield of purified exosomes [307] is approximately 4E12 exosomes in about 1 mL aqueous suspension.

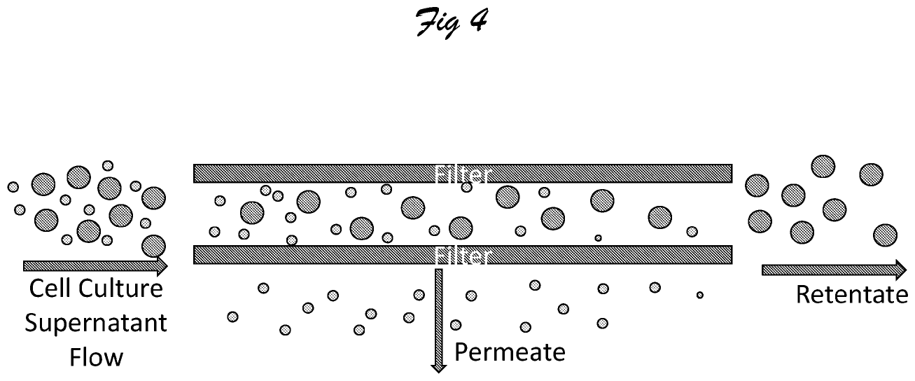

In one embodiment, the method is scalable exosome isolation process. FIG. 3 summarizes the scalable process [310]. Here, a 4-liter cell culture [311] is cultured and exosomes are produced and secreted into the culture medium (exosome production). Cells and cell debris are then removed from the culture medium by centrifugation followed by membrane filtration [313]. The exosomes are concentrated from the culture media sans cells and cell debris by tangential flow filtration (TFF) [314] in which the exosomes are retained in the retentate (see also FIG. 4). In some embodiments, TFF is performed in a “daisy-chain” manner in which the retentate from a first TFF step is subjected to a second (or additional) TFF step(s) to increase concentration and yield of exosomes. Protein is then removed from the exosome concentrate (TFF retentate) by size exclusion chromatography [315]. After protein removal, the exosomes are concentrated by TFF [316]. As in the first TFF step [314], TFF may be performed in a “daisy-chain” manner in which the retentate from a first TFF step is subjected to a second (or additional) TFF step(s) to increase concentration and yield of exosomes. Here, the yield of purified exosomes [317] is approximately 40E12 exosomes in about 13 mL aqueous suspension. Here, steps [311] through [315] may be repeated one or more additional times before feeding into the final TFF step [316].

In one embodiment, the method is a large-scale or GMP-ready exosomes isolation process. FIG. 3 summarizes the large-scale process [320]. Here, a 30-liter cell culture [321] is cultured and exosomes are produced and secreted into the culture medium (exosome production). Cells and cell debris are then removed from the culture medium by centrifugation [322] followed by membrane filtration [323]. The exosomes are concentrated from the culture media sans cells and cell debris by TFF [324] in which the exosomes are retained in the retentate (see also FIG. 4). In some embodiments, TFF is performed in a “daisy-chain” manner in which the retentate from a first TFF step is subjected to a second (or additional) TFF step(s) to increase concentration and yield of exosomes. Protein is then removed from the exosome concentrate (TFF retentate) by size exclusion chromatography [325]. After protein removal, the exosomes are concentrated by TFF [326]. As in the first TFF step [324], TFF may be performed in a “daisy-chain” manner in which the retentate from a first TFF step is subjected to a second (or additional) TFF step(s) to increase concentration and yield of exosomes. Here, the yield of purified exosomes [327] is approximately 140E12 exosomes in about 100 mL aqueous suspension. Here, steps [321] through [325] may be repeated one or more additional times before feeding into the final TFF step [326].

In some embodiments, the exosomes are native exosomes produced by primary cells, such as e.g., cardiosphere-derived cells (CDCs). In some embodiments, the exosomes are engineered exosomes produced from engineered cells. In certain particular embodiments, the engineered cells are engineered 293 cells and the engineered exosomes derived-therefrom contain engineered fusion or chimeric proteins. In a specific embodiment, the engineered fusion or chimeric protein is a tetraspannin-polypeptide-containing fusion protein, such as e.g., a SARS-Cov-2 spike-CD9 fusion protein (STX-S) and or a SARS-Cov-2 nucleocapsid-CD9 fusion protein, or a VHH or ScFv-CD9 fusion protein that provides for the external display of the non-tetraspannin protein of the fusion/chimeric protein on the exosome.

In one example of a lab-scale or scalable exosomes isolation process (see e.g., FIG. 3, lab-scale process [300], and scalable process [310]) HEK293 producer cells transfectedwith and producing a SARS-COV-2 spike-CD9 fusion protein (a.k.a. STX-S cells) were cultured in FREESTYLE media (ThermoFisher, 12338018) for 3 days in a Multitron incubator (Infors HT) at 37° C., 80% humidified atmosphere with 8% CO₂ on an orbital shaker platform rotating at 110 rpm. Subsequently, cells and cell debris were removed by low-speed centrifugation at 300×g for 5 min, followed by 3000×g for 15 min, while microvesicles and other extracellular vesicles larger than ~220 nm were removed by vacuum filtration of the conditioned media supernatant across an ~220 nm pore size diameter sterile filter. The resultant material is a sterile-filtered, clarified tissue culture supernatant (CTCS) with a volume roughly equal to the final culture volume. Next, exosomes were isolated from the CTCS using either a lab scale or a large-scale purification method. For lab scale: CTCS was subjected to concentrating filtration against a Centricon Plus-70 Centrifugal filter unit with a 100 kDa pore size membrane (UFC710008, Millipore, Burlington, MA) to generate a concentrated vesicle suspension (CVS). This was then subjected to size exclusion chromatography (SEC) using a qEV original SEC column (SP5, Izon, Christchurch, NZ). Exosome-containing fractions were further concentrated using 100 kDa Amicon centrifugal filters (Millipore, UFC810096) to produce purified exosomes. For scalable process: CTCS was subjected to concentrating tangential flow filtration (TFF) against a 750 kDa TFF filter (D06-E750-10-S, Repligen, Waltham, MA) on an AKTA Flux s instrument (Cytiva, Marlborough, MA) to generate a concentrated vesicle suspension (CVS). This was then subjected to size exclusion chromatography (SEC) using HiPrep 26/60 Sephacryl S-400 HR column (Cytiva, 28935605) on an AKTA Avant 25 (Cytiva, United States). Exosome-containing fractions were further concentrated using a 750 kDa TFF filter (Cytiva, UFP-750-E-3MA) on an AKTA Flux instrument (Cytiva, United States) to produce purified exosomes.

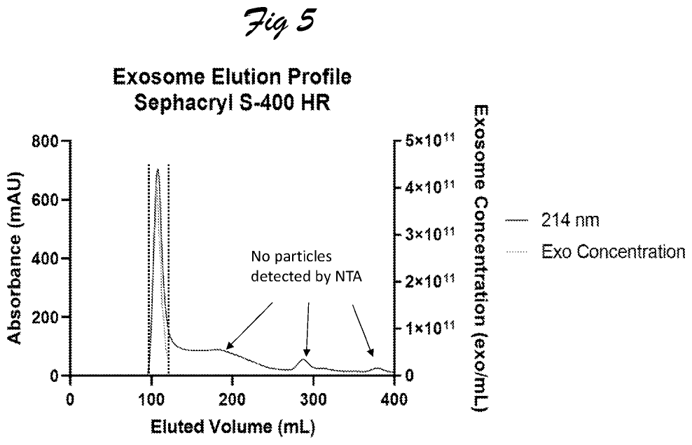

In one embodiment, protein and other non-exosomal contaminants present in the initial clarified cell culture media or concentrated (by TFF or other filtration method or other concentrating method) clarified cell culture media may be removed by size exclusion chromatography (SEC). In one embodiment, a Sephacryl media may be used, such as, e.g., Sepacryl S-400 high resolution chromatography in an AKTA Avant 25 system for example. FIG. 5 demonstrates an elution profile of exosomes coming off SEC in the initial flow-through volume. Here, the exosome-positive peak (as determined by Nanoparticle Tracking Analysis) aligned with first protein absorbance peak at around 100 mL eluted volume.

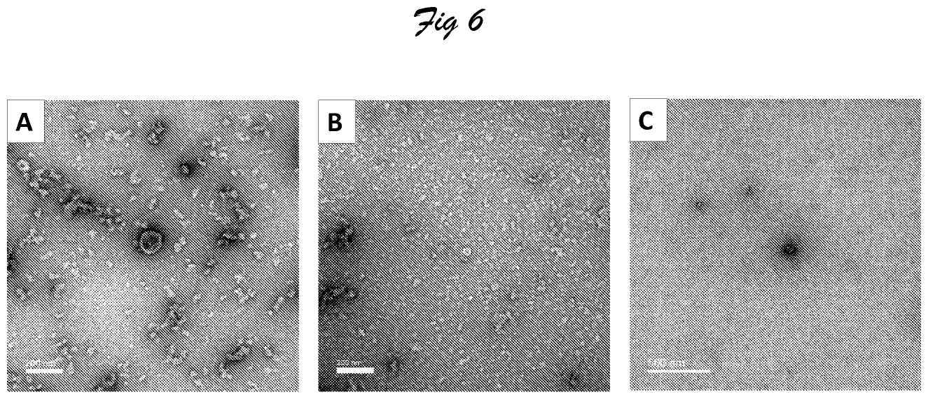

Here, the first pooled protein peak was demonstrated to contain >100 nm particles detected by NTA (signifying exosomes), exosome structure seen in TEM (FIG. 6, panel A), the presence of CD81, CD9, HSP60 by Western blot, and the absence of GM130/Calnexin/Cyt C by Western blot. The second pooled peak (first non-flow-through peak) was demonstrated to contain some 50-60 nm circular particles and undefined 40 nm material, (FIG. 6, panel B), and HSP60 protein by Western blot; and not to contain particles detectable by NTA, tetraspannins CD81 or CD9 by Western blot, and GM130, calnexin, or Cyt C detectable by Western blot. Pooled peaks 3 and later were demonstrated to be devoid of any particles by TEM (FIG. 6, panel C), and devoid of CD81, CD9, HSP60, GM130, calnexin, and Cyt C.

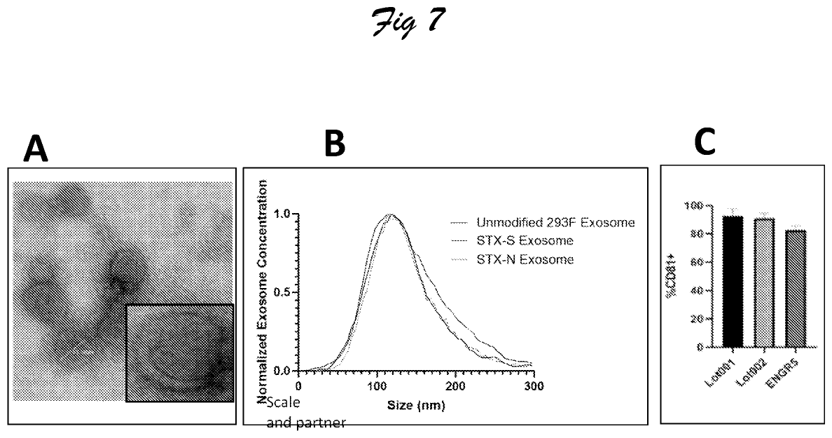

In one embodiment in which the exosomes are 293-derived exosomes expressing SARS-CoV-2 spike-CD9 fusion protein (STX-S) and/or 293-derived exosomes expressing SARS-COV-2 nucleocapsid-CD9 fusion protein (STX-N) and isolated, purified and concentrated via a large-scale or GMP-ready process (e.g., FIG. 3, process [320]), the final exosome product [327] was demonstrated by transmission electron microscopy (FIG. 7A) to contain exosomes, having a size distribution mean between 100 and 200 nm diameter (FIG. 7B), and expressing the exosomal marker CD81 (FIG. 7C).

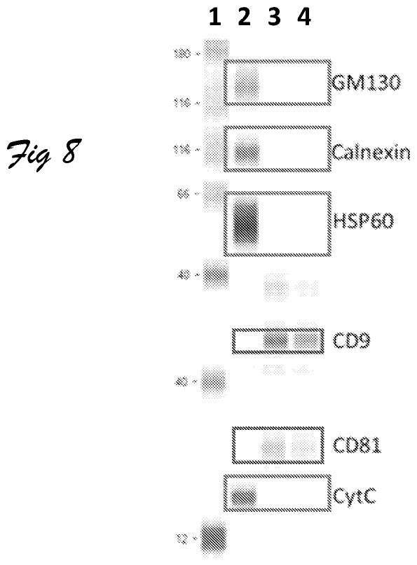

Here also, isolated, purified, and concentrated exosomes [307] [317] 327] were characterized by Western blot (JESS) (FIG. 8) and found to express or contain exosome-specific markers CD9 and CD81, but not the non-exosomal proteins GM130, HSP60, or CytC.

### Embodiments

Several embodiments are described.

Embodiment 1 is a method of producing vesicles, the method comprising obtaining a clarified cell culture supernatant containing vesicles and having a first volume; cross filtering the clarified cell culture supernatant to produce a first retentate containing said vesicles and having a second volume; sieving the first retentate; collecting a fraction containing said vesicles and having a third volume; and cross filtering the fraction containing vesicles and collecting a second retentate containing said vesicles and having a fourth volume.

Embodiment 2 is a method of embodiment 1 further comprising freezing and thawing the second retentate; centrifuging the thawed second retentate to produce a precipitate-free supernatant containing said vesicles; and filtering the precipitate-free supernatant to produce purified vesicles.

Embodiment 3 is a method of embodiment 1 or 2, wherein the clarified cell culture supernatant is produced by centrifuging a suspension cell culture to remove cells.

Embodiment 4 is a method of any one of embodiments 1-3, wherein the clarified cell culture supernatant is produced by centrifuging a suspension cell culture at a first force to produce a cell-free supernatant and then centrifuging the cell-free supernatant at a second force to produce the clarified cell culture supernatant.

Embodiment 5 is a method of embodiment 4, wherein the first force is 200-400×g.

Embodiment 6 is a method of embodiment 4 or 5, wherein the second force is 2,000-4,000×g.

Embodiment 7 is a method of any one of embodiments 1-6, wherein the clarified cell culture supernatant is cross filtered across a 750 kDa filter.

Embodiment 8 is a method of any one of embodiments 1-7, wherein the fraction containing vesicles is cross filtered across a 750 kDa filter.

Embodiment 9 is a method of any one of embodiments 1-8, wherein the first retentate is sieved over a size-exclusion matrix.

Embodiment 10 is a method of any one of embodiments 2-9, wherein the precipitate-free supernatant is filtered through a 0.2-micron filter.

Embodiment 11 is a method of any one of embodiments 1-10, wherein the purified vesicles have a diameter of 3-200 nm.

Embodiment 12 is a method of any one of embodiments 1-11, wherein the purified vesicles are exosomes.

Embodiment 13 is a method of any one of embodiments 1-12, wherein the second retentate comprises ≥1E12 vesicles per milliliter.

Embodiment 14 is a method of any one of embodiments 1-13, wherein the clarified cell culture supernatant contains vesicles secreted by cells, wherein the cells are cardiosphere-derived cells or HEK 293 cells.

Embodiment 15 is a method of any one of embodiments 1-14, wherein the clarified cell culture supernatant contains vesicles secreted by cells, wherein the cells are engineered to contain a cargo.

Embodiment 16 is a method of any one of embodiments 1-15, wherein the clarified cell culture supernatant contains vesicles secreted by cells, wherein the cells are engineered to express a targeting moiety.

Embodiment 17 is a method of any one of embodiments 1-14, wherein the clarified cell culture supernatant contains vesicles secreted by cells, wherein the cells are not engineered.

Embodiment 18 is a method of any one of embodiments 1-17, wherein the second volume is 40-75-fold smaller than the first volume.

Embodiment 19 is a method of any one of embodiments 1-18, wherein the fourth volume is 100-400-fold smaller than the first volume.

Embodiment 20 is a method of any one of embodiments 2-19, wherein the purified vesicles have a concentration of 1E11-1E13 vesicles/mL.

Embodiment 21 is exosomes produced according to the method of any one of embodiments 1-20.

Embodiment 22 is exosomes of embodiment 21, wherein the exosomes express or contain CD9.

Embodiment 23 is exosomes of embodiment 21 or 22, wherein the exosomes express or contain CD81.

Embodiment 24 is exosomes of any one of embodiments 21-23, wherein the exosomes do not express or contain GM130.

Embodiment 25 is exosomes of any one of embodiments 21-24, wherein the exosomes do not express or contain calnexin.

Embodiment 26 is exosomes of any one of embodiments 21-25, wherein the exosomes do not express or contain CytC.

Embodiment 27 is a composition comprising exosomes at a concentration of 1E12 to 5E12 exosomes per milliliter aqueous suspension.

Embodiment 28 is a method of any one of embodiments 1-20, wherein the second retentate is freeze dried or spray dried.

Embodiment 29 is a method of any one of embodiments 1-2 and 28, wherein the second retentate is freeze dried or spray dried and resuspended in a volume that is 0.5×, 0.2×, 0.1×, or 0.01× volume of the fourth volume.

### EXAMPLES

### Example 1: GMP Process

In one embodiment, a GMP isolation and purification process as outlined in FIG. 3, steps 320, was performed using the following parameters.

### First Tangential Flow Filtration Step

An AKTA Flux™ 6 tangential flow filtration instrument (Cytiva Life Sciences) was used. For the first TFF step 324 performed on the cell culture supernatant, the instrument was fitted with a polysulfone membrane filter having a 1 mm fiber inner diameter, 63.5 cm length, 750,000 nominal molecular weight cutoff and a surface area of 30.28 square meters, i.e., Cytiva/UFP-750-E-6A (Cytiva Life Sciences).

The system was primed with 0.1 μm filtered 0.1× or 1 PBS. The reservoir was charged with 2 L 0.1 μm filtered 0.1× or 1×PBS. The stirrer was set to 100 RPM. The feed pump was set at 2 L/min and the pressure adjusted using the retentate clamp so that the TMP was between 2.5-3 psi. The permeate port was opened to ensure that liquid was flowing into the permeate waste container and run for 1 minute or until bubbles were removed from the system. The permeate port was then closed and the TMP was adjusted to or maintained at 2.5-3 psi. 0.1× or 1×PBS was then circulated through the system for 6 minutes.

Approximately 7-8 psi of pressure was gently applied to the system using the retentate clamp for 4 minutes to fully saturate the membrane pores. The clamp on the permeate tubing was moved down to be closer to the ground to allow more room for liquid to collect in the tubing. In some cases, the permeate port tubing connection may drip slightly, but any forceful stream of liquid out of the port indicates that the permeate tubing is not properly attached. In some cases, it is necessary to decrease pressure over a 4-minute span.

After 4 minutes had elapsed, the pressure was released from the retentate clamp, and the permeate port was opened. About 500 mL of 0.1× or 1× PBS was allowed to drain through the permeate port, which was subsequently closed. The lower drain port was then opened, and the reservoir drained. Once drained, the lower drain port was closed.

500 mL PBS was added, and the flow rate set at 2 L/min, with the drain port and permeate port closed, and circulated for 1 minute. The reservoir was then drained through the top drain port, which was subsequently closed.

1 L PBS was added, and the flow rate set at 2 L/min. Pressure was applied using the retentate clamp so that the TMP was between 2-3 psi, and the PBS was circulated for 1 minute with both ports closed. The permeate port was then opened, the pump run with the port open for 1 minute, and the initial permeate flow rate was measured by collecting permeate for 1 minute in a 250 mL graduated cylinder. The feed pump was then stopped to ensure that some PBS remained in the system.

Aliquots of each bottle of clarified tissue culture supernatant (CTCS) were labeled with sub-lot ID, initials, cell line of origin, and date: 50 μL CTCS (store at −80° C.) was used for BCA assay; 50 μL CTCS (store at −80° C.) was used for nanoparticle tracking analysis (NTA) (ZETA VIEW, Particle Metrix GmbH, Inning am Ammersee, DE).

The volume of CTCS from each bottle was measured before adding it to the reservoir, which can hold up to 8 L at a time. The stir bar was set to 100 RPM and the feed pump was set to 2 L/min. The permeate was port is opened and the CTCS was added. This process was repeated until all CTCS had been added to the reservoir. The flow rate was measured after every approximately 3 L of CTCS was added. The filter can process up to 18 L of CTCS.

The retentate was concentrated to the minimum volume of the tubing as follows. As the volume of the reservoir decreased to the sloped bottom of the reservoir, the TMP reading on the display was monitored. The TMP drops rapidly, to below ~1.6 psi, and bubbles will appear in the FIL tubing leading from the lowest part of the tank.

2 L of 0.1 μm filtered 0.1× or 1×PBS was then to the reservoir and the retentate

concentrated to the minimum volume of tubing. Once the retentate reached the minimum volume of the tubing, the permeate port was closed and the retentate was recirculated for 30 minutes. The top drain port was opened and the retentate was collected in a 500 ml conical tube.

500 mL of 0.1 μm filtered 0.1× or 1×PBS was then added to the reservoir and the sides of the reservoir were rinsed. The retentate was concentrated to the minimum volume of tubing. Once the retentate reached the minimum volume of the tubing, the permeate port was closed and the retentate was recirculated for 30 minutes. The top drain port was opened and the retentate was collected in the same 500 ml conical tube as the first retentate.

The retentate sample was centrifuged at 4,000×g for 1 hour and the supernatant transferred to a new 500 ml conical tube. When the collected volume was less than 400 mL, the retentate was diluted to 400 mL with 0.1 μm filtered 0.1× or 1×PBS. Aliquots of the retentate were saved: 50 μL (store at −80° C.) was used for BCA assay; 50 μL (store at −80° C.) for nanoparticle tracking analysis using ZETAVIEW.

### Size Exclusion Chromatography

A column with an inner diameter of 100 mm and a bed height of 500 mm, i.e., AxiChrom 100/500 column (Cytiva Life Sciences, Marlborough, MA) was packed with a cross-linked copolymer of allyl dextran and N,N′-methylene bisacrylamide, i.e., Sephacryl S-400 HR resin (Cytiva Life Sciences) to 50-60 cm bed height and equilibrated to pH 7-7.5. Equilibration was performed by running 1×PBS or 0.1×PBS through the column.

The amount of solution to run the exosomes over the column varied slightly depending on the actual column volume, which ranged from 3926 mL to 4712 mL depending on the packed bed height. The ranges of solutions needed for one Elution+CIP cycle were 0.1× or 1×PBS: 3.7 CV (14.5 L-17.4 L), Water: 1 CV (3.9L-4.7L); 0.2 M Sodium Hydroxide: 0.5 CV (2.0 L-2.4 L).

Solutions or suspensions containing exosomes were loaded, eluted, and collected according to the conditions outlined in table 1. The method took about 2 hours to complete. Once the method finished, the chromatogram was reviewed for quality. Here, the pH during sample elution should be stable at approximately 7.2-7.5, and the system flow rate should be at 18 cm/hr while sample was eluting. If the system flow rate drops, the sample's elution will be inconsistent.

TABLE 1
Method Details

| | |
|---|---|
| Equilibration: | 0.10 CV of 1X PBS or 0.1X PBS, flow rate 29 cm/hr |
| Sample Application: | Flow Rate: 18 cm/hr |
| | 0.05 CV, Inlet primed before use with 17 mL for the first sample run o |
| Conditional | Flow Rate: 18 cm/hr |
| Fractionation | Watch for UV₂₅₄/UV₂₁₄ ratio is below 0.2 |
| Elution: | Collect 0.07 CV Sample after UV₂₅₄/UV₂₁₄ ratio is below 0.2 |

Aliquots of the SEC-processed exosome sample were labeled. 200 μL of the SEC-processed sample was reserved for BCA or μBCA assay. 50 μL of the SEC-processed sample was reserved for nanoparticle tracking analysis (NTA) and Zeta View measurement. The sample was capped and either used immediately or stored at −80° C.

### Second Tangential Flow Filtration Step

An AKTA Flux™ 6 tangential flow filtration instrument (Cytiva Life Sciences) was used. For the second TFF step 326 performed on the exosome fraction from the size exclusion step 325, the instrument was fitted with a polysulfone membrane filter having a 1 mm fiber inner diameter, 33.7 cm length, and a nominal molecular weight cutoff of 750,000, i.e., Cytiva/UFP-750-E-3MA filter (Cytiva Life Sciences).

The system was primed with 0.1 μm filtered 0.1× or 1×PBS. The reservoir was charged with 2 L 0.1 μm filtered 0.1× or 1×PBS. The stirrer was set to 100 RPM. The feed pump was set at 2 L/min and the pressure adjusted using the retentate clamp so that the TMP was between 2.5-3 psi. The permeate port was opened to ensure that liquid was flowing into the permeate waste container and run for 1 minute or until bubbles were removed from the system. The permeate port was then closed and the TMP was adjusted to or maintained at 2.5-3 psi. 0.1× or 1×PBS was then circulated through the system for 6 minutes.

Approximately 12-13 psi of pressure was gently applied to the system using the retentate clamp for 4 minutes to fully saturate the membrane pores. The clamp on the permeate tubing was moved down to be closer to the ground to allow more room for liquid to collect in the tubing. In some cases, the permeate port tubing connection may drip slightly, but any forceful stream of liquid out of the port indicates that the permeate tubing is not properly attached. In some cases, it is necessary to decrease pressure over a 4-minute span.

After 4 minutes had elapsed, the pressure was released from the retentate clamp, and the permeate port was opened. About 500 mL of 0.1× or 1×PBS was allowed to drain through the permeate port, which was subsequently closed. The lower drain port was then opened, and the reservoir drained. Once drained, the lower drain port was closed.

500 mL PBS was added, and the flow rate set at 2 L/min, with the drain port and permeate port closed, and circulated for 1 minute. The reservoir was then drained through the top drain port, which was subsequently closed.

1 L PBS was added, and the flow rate set at 2 L/min. Pressure was applied using the retentate clamp so that the TMP was between 2.5-3 psi, and the PBS was circulated for 1 minute with both ports closed. The permeate port was then opened, the pump run with the port open for 1 minute, and the initial permeate flow rate was measured by collecting permeate for 1 minute in a 250 mL graduated cylinder. The feed pump was then stopped to ensure that some PBS remained in the system.

The stir bar was set to 100 RPM and the feed pump was set to 2 L/min. The permeate port was opened and the SEC fraction was added. This process was repeated until all SEC fractions had been added to the reservoir. The flow rate was measured every 10 minutes for the first 30 minutes, and every 30 minutes afterwards.

The retentate was concentrated to the minimum volume of the tubing as follows. As the volume of the reservoir decreased to the sloped bottom of the reservoir, the TMP reading on the display was monitored. The TMP drops rapidly, to below ~1.6 psi, and bubbles will appear in the FIL tubing leading from the lowest part of the tank. Once the retentate reached the minimum volume of the tubing, the permeate port was closed and the retentate was recirculated for 30 minutes. The top drain port was opened and the retentate was collected in a 225 ml conical tube.

500 mL of 0.1 μm filtered 0.1× or 1× PBS was then added to the reservoir and the sides of the reservoir were rinsed. The retentate was concentrated to the minimum volume of tubing. Once the retentate reached the minimum volume of the tubing, the permeate port was closed and the retentate was recirculated for 30 minutes. The top drain port was opened and the retentate was collected in a separate 225 ml conical tube.

The exosome concentrate was frozen and thawed.

The exosome concentrates were centrifuged at 3,000×g for 15 minutes at 4° C. hour and the supernatant transferred to a new conical tube. Aliquots of the final exosome concentrate were saved: 3×1 mL in 2 mL CZ vials (store at −80° C.) for retain and analysis; as many as possible×10 mL CZ vials (store at −80° C.) for use.

### Example 2: Large Scale Process

In one embodiment, a large-scale isolation and purification process as outlined in FIG. 3, steps 310, was performed using the following parameters.

### First Tangential Flow Filtration Step

An ÄKTA Flux™ s tangential flow filtration instrument (29038437, Cytiva Life Sciences) was used. For the first TFF step 314 performed on the cell culture supernatant, the instrument was fitted with a modified polyether-sulfone membrane filter having a 1 mm fiber inner diameter, 65 cm effective length, 750,000 nominal molecular weight cutoff and a surface area of 245 square centimeters, i.e., Repligen/D06-E750-10-S (Repligen, Waltham, MA).

The system was primed with 0.1 μm filtered 0.1× or 1×PBS. The reservoir was charged with 500 mL 0.1 μm filtered 0.1× or 1×PBS. The stirrer was set to 100 RPM. The feed pump was set at 80 rpm, the permeate port was opened to ensure that liquid was flowing into the permeate waste container, and the pump run for 1 minute or until bubbles were removed from the system. The permeate port was then closed and the feed pump was set to 55 rpm. 0.1× or 1×PBS was then circulated through the system for 6 minutes.

Approximately 12-13 psi of pressure was gently applied to the system using the retentate clamp for 4 minutes to fully saturate the membrane pores and ensure that there were no leaks in the system. After 4 minutes had elapsed, the pressure was released from the retentate clamp, and the permeate port was opened to drain the reservoir and then closed.

100 mL PBS (0.1× or 1×) was added, and the flow rate set 80 rpm, with the drain port and permeate port closed, and circulated for 1 minute. The reservoir was then drained through the drain port, which was subsequently closed.

150 mL PBS (0.1× or 1×) was added, and the flow rate set at 80 rpm. The PBS was circulated for 1 minute with both ports closed. The permeate port was then opened, the pump run with the port open for 1 minute, and the initial permeate flow rate was measured by collecting permeate for 1 minute in a 50 ml conical tube, or by collecting permeate for 30 seconds and then multiplying the volume collected by 2. The feed pump was then stopped to ensure that some PBS remained in the system.

Aliquots of each bottle of clarified tissue culture supernatant (CTCS) were labeled with sub-lot ID, initials, cell line of origin, and date: 50 μL CTCS (store at −80° C.) was used for BCA assay; 50 μL CTCS (store at −80° C.) was used for nanoparticle tracking analysis (NTA) (ZETA VIEW, Particle Metrix GmbH, Inning am Ammersee, DE).

A bottle of CTCS was added to the reservoir by serological pipette and the volume was recorded. The stir bar was set to 100 RPM and the feed pump was set to 80 rpm. The permeate port was opened and the CTCS was concentrated. When the retentate volume in the reservoir reached 100 mL, the permeate flow rate was measured. When the permeate flow rate was above 5 mL/min, approximately 400 mL of well mixed CTCS was added to the reservoir. No additional CTCS was added when the permeate flow rate was below 5 mL/min. This process was repeated until all CTCS had been added to the reservoir. The filter can process up to 2 L of CTCS.

The retentate was concentrated to the minimum volume of the tubing as follows. The feed pump was run at 80 rpm until approximately 20 g of retentate remained in the reservoir. This is approximately the level at which the liquid reaches the R2L on the right side of the reservoir. The feed pump speed was lowered to 30 rpm. The concentrating of the retentate was continued until bubbles appeared in the FIL tubing leading to the peristaltic pump (approximately 6 g by the scale in the system). This is the minimum volume of the tubing and the filter.

200 mL of 0.1 μm filtered 0.1× or 1×PBS was then to the reservoir and the retentate concentrated to the minimum volume of tubing. Once the retentate reached the minimum volume of the tubing, the permeate port was closed and the retentate was recirculated for 30 minutes with the feed pump at 30 rpm. Tubing was removed from the drain port and the retentate was collected in the same 50 ml conical tube as the first retentate.

The retentate sample was centrifuged at 4,000×g for 1 hour and the supernatant transferred to a new 50 mL conical tube. When the collected volume was less than 42.5 mL, the retentate was diluted to 42.5 mL with 0.1 μm filtered 0.1× or 1×PBS. Aliquots of the retentate were saved: 50 μL (store at −80° C.) was used for BCA assay; 50 μL (store at −80° C.) for nanoparticle tracking analysis using ZETAVIEW.

### Size Exclusion Chromatography

A column with an inner diameter of 26 mm and a column length of 60 cm was packed with a cross-linked copolymer of allyl dextran and N,N′-methylene bisacrylamide, i.e., HiPrep™ 26/60 Sephacryl S-400 HR Column (#28935605, Cytiva) and run on the ÄKTA Avant 25 system (Cytiva).

Here, each elution processed13 mL of the TFF-CVS (“concentrated vesicle supernatant,” i.e., concentrated exosomes from the first TFF step 314). The details of the run are summarized in table 2.

TABLE 2
Method Details

| | |
|---|---|
| Equilibration: | 0.10 CV of 0.1X or 1X PBS, line A1, flow rate 2.6 mL/min |
| Sample Application: | Flow Rate: 1.6 mL/min |
| | 13 mL, prime Inlet before use with 3.5 mL for the first run of the |
| | sample |
| Elution: | Elution with 0.1X or 1X PBS, line A1, flow rate 1.6 mL/min |
| | Isocratic Elution 0.50 column volumes (CV), start after 0.15 CV |
| | Fixed volume fractionation, 1.5 mL each in 96 deep well plate. |
| | OR |
| | Peak volume fractionation, based on the absorbance of a previously |
| | run sample. |
| Accumulator Wash: | 20 mL H₂O, line A2, flow rate 10 mL/min through fraction collector |
| | accumulator |
| Column CIP: | 1. 0.03 CV of 1X PBS, line A1, 2 mL/min flow rate, down flow |
| | 2. 0.03 CV of 1X PBS, line A1, 2 mL/min flow rate, up flow |
| | 3. 0.5 CV of 0.2M NaOH solution, line A3, 2 mL/min flow rate, up |
| | flow. |
| | 4. 0.5 CV of Water, line A2, 2 mL/min flow rate, up flow |
| | 5. 0.5 CV of 1X PBS, line A1, 2.6 mL/min flow rate, up flow, |
| | incubate 2 min |
| Equilibration: | 4 CV of 1X PBS, flow rate 2.6 mL/min, down flow |

Fractionation/elution steps may be fixed volume elution (FV) or peak volume elution (PV). For FV, the sample was eluted into a 96 deep-well plate at 1.5 mL per fraction. For PV, the sample was eluted into 50 ml conical tubes based on set absorbance levels at 214 nm. Fractions that absorb higher than 250 mAU were eluted into a 50 ml conical tube.

Samples were collected in a refrigerated fraction collector. In the evaluation window in UNICORN™ (Cytiva), the contents of each collected fraction were cross-referenced with the variables collected during the run. Those variable of interest during the run are: pH, conductivity, UV Absorbance at 214 nm, and system flow rate.

Before collecting the sample, to ensure its quality: pH during sample elution was stable at approximately 7.4; conductivity during sample elution was stable at approximately 17.2 mS/cm; sample showed a peak in UV absorbance (214 nm) around 105 mL eluted volume; the pH at the end of the run was stable at approximately 7.4; conductivity at the end of the run was stable at approximately 17.2 mS/cm; system flow rate was 1.6 mL/min while the sample was eluting.

The sample was collected by opening the fraction collector door. When the sample was collected in fixed volumes in a 96 deep well plate (FV), immediately cover the plate was immediately covered with a MicroAmp™ optical adhesive film (ThermoFisher, Waltham, MA). When the sample was processed into a 96 deep well plate, exosome containing fractions were pooled. Here, the exosome peak was around 105 mL eluted volume. The covered plate was transferred to a biosafety cabinet the exosome-containing fractions pooled in a 50 ml conical tube.

Aliquots of each run's SEC pooled fractions were saved: 200 μL pooled SEC fraction (store at −80° C.) used for BCA or μBCA assay; 50 μL pooled SEC fraction (store at −80° C.) used for NTA ZETAVIEW measurement. For those runs eluted into a 96 well deep-well plate, aliquots from the final well filled during the run were saved: 100 μL final well SEC fraction (store at −80° C.) used for NTA ZETAVIEW measurement.

### Second Tangential Flow Filtration Step

An AKTA Flux™ 6 tangential flow filtration instrument (Cytiva Life Sciences) was used. For the second TFF step 316 performed on the exosome fraction from the size exclusion step 315, the instrument was fitted with a polysulfone membrane filter having a 1 mm fiber inner diameter, 33.7 cm length, and a nominal molecular weight cutoff of 750,000, i.e., Cytiva/UFP-750-E-3MA filter (Cytiva Life Sciences).

The system was primed with 0.1 μm filtered 0.1× or 1×PBS. The reservoir was charged with 300 mL 0.1 μm filtered 0.1× or 1×PBS. The stirrer was set to 100 RPM. The feed pump was set at 125 rpm, the permeate port was opened to ensure that liquid was flowing into the permeate waste container, and the pump run for 1 minute or until bubbles were removed from the system. The permeate port was then closed and the feed pump was set to 88 rpm. 0.1× or 1×PBS was then circulated through the system for 6 minutes.

Approximately 12-13 psi of pressure was gently applied to the system using the retentate clamp for 4 minutes to fully saturate the membrane pores and ensure that there were no leaks in the system. After 4 minutes had elapsed, the pressure was released from the retentate clamp, and the permeate port was opened to drain the reservoir and then closed.

100 mL PBS (0.1× or 1×) was added, and the flow rate set 125 rpm, with the drain port and permeate port closed, and circulated for 1 minute. The reservoir was then drained through the drain port, which was subsequently closed.

150 mL PBS (0.1× or 1×) was added, and the flow rate set at 125 rpm. The PBS was circulated for 1 minute with both ports closed. The permeate port was then opened, the pump run with the port open for 1 minute, and the initial permeate flow rate was measured by collecting permeate for 1 minute in a 15 mL conical tube, or by collecting permeate for 30 seconds and then multiplying the volume collected by 2. The feed pump was then stopped to ensure that some PBS remained in the system.

To concentrate the SEC fractions containing exosomes, the stir bar was set to 100 RPM and the feed pump was set to 125 RPM. The permeate port was opened and the exosome-containing solution was loaded and concentrated. The permeate flow rate was measured every 10 minutes for the first 30 minutes, and every 30 minutes afterwards. This step was repeated until all SEC exosome-containing fractions had been added to the reservoir.

The retentate was concentrated to the minimum volume of the tubing. Here, the feed pump was run at 125 RPM until approximately 20 g of retentate remained in the reservoir. This is approximately the level at which the liquid reaches the R2L on the right side of the reservoir. The feed pump speed was then lowered to 44 RPM.

Concentrating the retentate was continued until bubbles appeared in the FIL tubing leading to the peristaltic pump (approximately 6 g by the scale in the system). This is the minimum volume of the tubing and the filter. Once the retentate reached the minimum volume of the tubing, the permeate port was closed and the retentate was recirculated for 30 minutes with the feed pump speed at 44 RPM. Any tubing attached to the drain port was then removed and the retentate collected in a 15 ml conical tube.

100 mL of 0.1 μm filtered 0.1× or 1×PBS was added to the reservoir and the retentate was concentrated to the minimum volume of tubing. Once the retentate reached the minimum volume of the tubing, the permeate port was closed and the retentate was recirculated for 30 minutes with the feed pump speed set at 44 RPM. Any tubing attached to the drain port was removed and the retentate was collected in a separate 50 ml conical tube.

The exosome concentrates were frozen and thawed then centrifuged at 3,000×g for 15 minutes at 4° C. The exosome concentrates were transferred to a new conical tube, leaving behind the pellet, and the volume of the retentate collected was measured.

Samples of final exosome concentrate were retained, labeled with date, initials, cell line, and containing: 3×50 μL (store at −80° C.) used for NTA ZETAVIEW measurements; 3×50 μL (store at −80° C.) used for BCA analysis; 1×200 μL (store at −80° C.) used for CD81 analysis; 1×250 μL (store at −80° C.) used for ELISA/JESS analysis (in the case of engineered exosomes); and 1×1 mL (store at −80° C.) used for retention.

### Example 3: Lab Scale Process

In one embodiment, a lab scale isolation and purification process as outlined in FIG. 3, steps 300, was performed using the following parameters.

The number of Centricon filters, SEC isolations, and Amicon filters were scaled based on the amount of clarified tissue culture supernatant (CTCS) 230 that needs to be processed (table 3)

TABLE 3

| | | Suggested # of | Suggested CVS | | Suggested # of |
|---|---|---|---|---|---|
| Volume of CTCS | | Centricons | Final Volume | | Amicons |
| 0-250 | mL | 1 | 1.5 | mL | 1 |
| 250-500 | mL | 2 | 3 | mL | 2 |
| 500-1250 | mL | 4 | 4.5 | mL | 3 |
| 1250-1800 | mL | 6-8 | 6 | mL | 4-6 |

Loading Centricon Plus-70 (100k) Filters with CTCS

Centricon® filters (EMD Millipore, Burlington, MA) were prepared in a biosafety cabinet (BSC). Each Centricon® filter can effectively filter about 125 mL CTCS. Here, six 350 mL bottles of CTCS was distributed across eight Centricon® filters.

The filter(s) were pre-rinsed with 60 mL of UltraPure DNase/RNase-Free distilled water, and then centrifuged at 3000×g for 5 min. The filters were then sterilized by adding 60 mL of 70% ethanol to the filter and letting it sit for 10 min centrifugation at 3000×g for 5 minutes. The filters were then washed once with 60 mL of UltraPure DNase/RNase-Free distilled water, then centrifuged at 3000×g for 5 minutes, then washed with 60 mL of 0.1 μm filtered 1×PBS, and then centrifuged at 3000×g for 5 minutes.

Throughout the procedure, the filters are not allowed to dry out.

A 50 μL aliquot of CTCS was set aside for NTA ZETAVIEW (stored on ice or −80° C.). A 50 μL aliquot of CTCS was set aside for BCA (stored on ice or −80° C.).

55 mL of CTCS was added to each filter, and centrifuged at 500×g for 2 minutes, followed by 3000×g for 20 minutes. The filter volume was checked and spun until the volume reached about 3 mL in each filter. The flow through was discarded.

The remaining volume of CTCS was added to each filter in the BSC and centrifuged at 3000×g for 30 minutes with additional time as needed until the final volume reached about 500 μLs/filter. The flow through was discarded.

### Recover Clarified Vesicle Supernatant (CVS)

In the BSC, the retentate cup was sterilized with 90% ethanol and allowed to completely dry. The retentate cup was placed upside down on top of the filter cup, the filter cup was removed from flow-through cup, the filter/retentate cup was inverted to orient the filter on top and the retentate cup on bottom. The filter/retentate cup was placed in the centrifuge and centrifuged at 200×g for 2 minutes.

The filter and retentate cup were transferred to a BSC, keeping the filter cup inverted during the process. The CVS was removed and collected in a low retention Eppendorf tube or a 15 ml conical tube. Here,

2 mL of CTCS used four Centricon® filters or 4 mL per eight Centricon® filters. When the volume of CTCS is >1 mL above the expected volume, the CVS was put back on two Centricon® filters and continued to concentrate.

### Recover Exosomes Attached to Centricon Plus-70 Filter Membrane

The bottom of the filters was sealed with parafilm (paper side in). 3 mL of 0.1 μm filtered 1×PBS was added to each Centricon filter cup (1.5 mL per post). The filter was then capped with concentrate/retentate cup. The filter column was vortexed 20 seconds to release the particles attached to membrane. Material was recovered from all but two filters, e.g., if eight filters were used in total, the recovered material from six filters is collected. The

The retentate cup was placed on top of filter cup, the flow-through cup was removed, inverted, and centrifuged at 200×g for 2 minutes. The collected volume was added to the two remaining uncollected filters, centrifuged at with 500×g for 2 minutes, then centrifuged at 3000×g for 10 minutes. In some cases, additional time was needed.

When the final volume reached around 500 μL in each filter, the recovered concentrate was collected from the 2 filters (~1 mL total) by placing the retentate cup on top of filter cup, removing the flow-through cup, inverting, and centrifuging at 200×g for 2 minutes. The collected volume was added to the previously collected CVS concentrate. When necessary, the CVS was diluted with PBS to collect a final volume based on the suggested CVS final volume disclosed in table 3.

Run CVS Material on qEV original Size Exclusion Columns

The CVS material 245 was subjected to qEV SEC 35 nm columns (Izon, Scottsdale, AZ).

2 mL collection tubes were labeled with desired fractions to collect as described in table 4.

TABLE 4

| Fraction Number | Eluted Volume (mL) | Contains Exosomes? |
|---|---|---|
| 1 | 0-0.5 | No |
| 2 | 0.5-1 | No |
| 3 | 1-1.5 | No |
| 4 | 1.5-2 | No |
| 5 | 2-2.5 | No |
| 6 | 2.5-3 | No |
| 7 | 3-3.5 | YES |
| 8 | 3.5-4 | YES |
| 9 | 4-4.5 | YES |
| 10 | 4.5-5 | YES |
| 11 | 5-5.5 | Yes, with protein contam. |
| 12 | 5.5-6 | No |

0.5 mL of sample (CVS) was loaded onto each column loading frit. Once the liquid reached the loading frit, 2.5 mL of 0.1 μm filtered 1×PBS was added to elute fractions 2-6 (the void volume). 2 mL of 0.1 μm filtered 1×PBS was then added to the column to collect fractions 7-10 in one tube. Alternatively, 0.5 mL 0.1 μm filtered 1×PBS was added four times to collect fractions 7-10 separately. To collect accurate volumes, only the required volume was added to the top of the column.

Once the first 0.5 mL of CVS had been fully fractionated, the column was flushed with 10 mL 0.5 M NaOH followed 20 mL of 0.1 μm filtered 1×PBS.

The collection was repeated with the remaining CVS, with flushing performed between collections with NaOH and 0.1 μm filtered 1×PBS. Collected fractions were stored at −80° C.

Combine Exosome Positive Fractions using Amicon Filter ULTRA-4 (100K-4 ml)

A number of Amicon® filters (EMD Millipore) were prepared based on the suggested number from table 3. In the BSC, filters were (i) pre-rinsed by adding 4 mL of UltraPure DNase/RNase-Free distilled water, then centrifuged at 3000×g for 5 minutes at 4° C., (ii) sterilized by adding 4 mL of 70% ethanol to the filter and letting it sit for 10 minutes, then centrifuged at 3000×g for 5 minutes at 4° C., (iii) washed once with 4 mL of UltraPure DNase/RNase-Free distilled water, then centrifuged at 3000×g for 5 minutes at 4° C., and (iv) washed with 4 mL 0.1 μm filtered 1×PBS, then centrifuged at 3000×g for 5 minutes at 4° C.

4 mL exosome-containing fractions was added to each filter, centrifuged at 500×g at 4° C. for 2 min followed with centrifugation for 20 min at 3000×g.

Exosome-containing fractions were continued to be added (up to 4 mL per filter) followed by centrifuging at 3000×g at 4° C. as needed. When the volume reached about 250 μL per filter, the concentrate was recovered using a 200 μL pipette (low retention tips). The sample was transferred to a low retention Eppendorf tube or 15 mL conical tube (or larger as needed).

After collecting the concentrate, the bottom of filter was sealed with parafilm. 750 μL of 0.1 μm filtered 1×PBS was added to each filter, and each filter vortexed for 10 seconds to release the particles from the membrane. The parafilm was removed from the filters and the filters were centrifuged at 3000×g at 4° C. until volume reached about 250 μL per filter. 200 μL of 0.1 μm filtered 1×PBS was added to each filter and pipetted into the exosome sample to ensure full recovery.

The recovered purified exosome product (PEP) was frozen at −80° C. and thawed on ice. In a BSC, the PEP was transferred into sterile microcentrifuge tubes, centrifuged at 10,000×g for 10 minutes at 4° C. In a BSC, 1 mL PEP supernatant was transferred to new sterile microcentrifuge tubes and the total volume collected was recorded.

Aliquots for characterization assays were prepared: 100 μL PEP for CD81, 50 μL PEP for BCA assay, and 50 μL PEP for NTA ZETAVIEW. The remainder was stored in 1 mL aliquots in microcentrifuge tubes, and labeled accordingly including cell line, lot ID, date, and initials. The assay samples and 1 mL PEP aliquots were stored at −80° C.

Although the foregoing has been described in some detail by way of illustrations and examples for purposes of clarity and understanding, it will be understood by those of skill in the art that modifications can be made without departing from the spirit of the present disclosure. Therefore, it should be clearly understood that the forms disclosed herein are illustrative only and are not intended to limit the scope of the present disclosure, but rather to also cover all modification and alternatives coming with the true scope and spirit of the embodiments of the invention(s).

It is contemplated that various combinations or subcombinations of the specific features and aspects of the embodiments disclosed above may be made and still fall within one or more of the inventions. Further, the disclosure herein of any particular feature, aspect, method, property, characteristic, quality, attribute, element, or the like in connection with an embodiment can be used in all other embodiments set forth herein. Accordingly, it should be understood that various features and aspects of the disclosed embodiments can be combined with or substituted for one another in order to form varying modes of the disclosed inventions. Thus, it is intended that the scope of the present inventions herein disclosed should not be limited by the particular disclosed embodiments described above. Moreover, while the invention is susceptible to various modifications, and alternative forms, specific examples thereof have been shown in the drawings and are herein described in detail. It should be understood, however, that the invention is not to be limited to the particular forms or methods disclosed, but to the contrary, the invention is to cover all modifications, equivalents, and alternatives falling within the spirit and scope of the various embodiments described and the appended claims. Any methods disclosed herein need not be performed in the order recited. The methods disclosed herein include certain actions taken by a practitioner; however, they can also include any third-party instruction of those actions, either expressly or by implication. For example, actions such as “administering an antigen-binding protein” include “instructing the administration of an antigen-binding protein.” In addition, where features or aspects of the disclosure are described in terms of Markush groups, those skilled in the art will recognize that the disclosure is also thereby described in terms of any individual member or subgroup of members of the Markush group.

The ranges disclosed herein also encompass any and all overlap, sub-ranges, and combinations thereof. Language such as “up to,” “at least,” “greater than,” “less than,” “between,” and the like includes the number recited. Numbers preceded by a term such as “about” or “approximately” include the recited numbers. For example, “about 90%” includes “90%.” In some embodiments, at least 95% homologous includes 96%, 97%, 98%, 99%, and 100% homologous to the reference sequence. In addition, when a sequence is disclosed as “comprising” a nucleotide or amino acid sequence, such a reference shall also include, unless otherwise indicated, that the sequence “comprises”, “consists of” or “consists essentially of” the recited sequence.

Terms and phrases used in this application, and variations thereof, especially in the appended claims, unless otherwise expressly stated, should be construed as open ended as opposed to limiting. As examples of the foregoing, the term ‘including’ should be read to mean ‘including, without limitation,’ ‘including but not limited to,’ or the like.

The indefinite article “a” or “an” does not exclude a plurality. The term “about” as used herein to, for example, define the values and ranges of molecular weights means that the indicated values and/or range limits can vary within ±20%, e.g., within ±10%. The use of “about” before a number includes the number itself. For example, “about 5” provides express support for “5”. Numbers provided in ranges include overlapping ranges and integers in between; for example a range of 1-4 and 5-7 includes for example, 1-7, 1-6, 1-5, 2-5, 2-7, 4-7, 1, 2, 3, 4, 5, 6 and 7.

1. A method of producing vesicles, the method comprising:
obtaining a clarified cell culture supernatant containing vesicles and having a first volume;
cross filtering the clarified cell culture supernatant to produce a first retentate containing said vesicles and having a second volume;
sieving the first retentate;
collecting a fraction containing said vesicles and having a third volume; and
cross filtering the fraction containing vesicles and collecting a second retentate containing said vesicles and having a fourth volume.

2. The method of claim 1 further comprising:
freezing and thawing the second retentate;
centrifuging the thawed second retentate to produce a precipitate-free supernatant containing said vesicles; and
filtering the precipitate-free supernatant to produce purified vesicles.

3. The method of claim 1 or 2, wherein the clarified cell culture supernatant is produced by centrifuging a suspension cell culture to remove cells.

4. The method of claim 1 or 2, wherein the clarified cell culture supernatant is produced by centrifuging a suspension cell culture at a first force to produce a cell-free supernatant and then centrifuging the cell-free supernatant at a second force to produce the clarified cell culture supernatant.

5. The method of claim 4, wherein the first force is 200-400×g.

6. The method of claim 4, wherein the second force is 2,000-4,000×g.

7. The method of claim 1 or 2, wherein the clarified cell culture supernatant is cross filtered across a 750 kDa filter.

8. The method of claim 1 or 2, wherein the fraction containing vesicles is cross filtered across a 750 kDa filter.

9. The method of claim 1 or 2, wherein the first retentate is sieved over a size-exclusion matrix.

10. The method of claim 2, wherein the precipitate-free supernatant is filtered through a 0.2-micron filter.

11. The method of claim 1 or 2, wherein the purified vesicles have a diameter of 3-200 nm.

12. The method of claim 1 or 2, wherein the purified vesicles are exosomes.

13. The method of claim 1 or 2, wherein the second retentate comprises ≥1E12 vesicles per milliliter.

14. The method of claim 1 or 2, wherein the clarified cell culture supernatant contains vesicles secreted by cells, wherein the cells are cardiosphere-derived cells or HEK 293 cells.

15. The method of claim 1 or 2, wherein the clarified cell culture supernatant contains vesicles secreted by cells, wherein the cells are engineered to contain a cargo.

16. The method of claim 1 or 2, wherein the clarified cell culture supernatant contains vesicles secreted by cells, wherein the cells are engineered to express a targeting moiety.

17. The method of claim 1 or 2, wherein the clarified cell culture supernatant contains vesicles secreted by cells, wherein the cells are not engineered.

18. The method of claim 1 or 2, wherein the second volume is 40-75-fold smaller than the first volume.

19. The method of claim 1 or 2, wherein the fourth volume is 100-400-fold smaller than the first volume.

20. The method of claim 1 or 2, wherein the purified vesicles have a concentration of 1E11-1E13 vesicles/mL.

21. Exosomes produced according to the method of claim 1 or 2.

22. The exosomes of claim 21, wherein the exosomes express or contain CD9.

23. The exosomes of claim 21, wherein the exosomes express or contain CD81.

24. The exosomes of claim 21, wherein the exosomes do not express or contain GM130.

25. The exosomes of claim 21, wherein the exosomes do not express or contain calnexin.

26. The exosomes of claim 21, wherein the exosomes do not express or contain HSP60.

27. The exosomes of claim 21, wherein the exosomes do not express or contain CytC.

28. A composition comprising exosomes at a concentration of 1E12 to 5E12 exosomes per milliliter aqueous suspension.
